(19) United States
(12) Patent Application Publication
(10) Pub. No.: US 2002/0019629 A1
(43) Pub. Date: Feb. 14, 2002

(54) Devices, systems and methods for transluminally and controllably forming intramyocardial channels in cardiac tissue

(75) Inventors: Ulrich Dietz, Wiesbaden (DE); Harold Darius, Mainz (DE); Olaf Eick, Willich (DE); Victor Duysens, Grevenbicht (NL)

Correspondence Address: Thomas F. Woods, Medtronic, Inc., MS LC 340, 710 Medtronic Parkway, Minneapolis, MN, 55432-5604, US

(73) Assignee: Medtronic, Inc.

(21) Appl. No.: 09/871,657

(22) Filed: Jun. 4, 2001

Related U.S. Application Data
(63) Continuation-in-part of application No. 09/453,096, filed on Dec. 2, 1999.
(62) Division of application No. 09/113,382, filed on Jul. 10, 1998, now abandoned.
(60) Provisional application No. 60/210,733, filed on Jun. 12, 2000.

(51) Int. Cl. A61B 18/14

(52) U.S. Cl. 606/41

(57) ABSTRACT

A medical system and corresponding method for forming intramyocardial channels in cardiac tissue of a patient's heart are disclosed. The channel forming system may be configured in two basic embodiments: (a) a first embodiment where a transluminal steering and delivery system comprises a guide catheter or sheath which functions largely apart from and independent from other components of channel forming system (with the notable exception of a means for extending and retracting a piercing means from a distal end of the guide catheter or sheath, and (b) a second embodiment where a transluminal steering and delivery system is integrated into and combined with other components of the channel forming system, such as a piercing means, means for delivering radio-frequency energy to the piercing means, and temperature sensing means.

### RELATED APPLICATIONS

This patent application claims priority and other benefits from U.S. Provisional Patent Application Ser. No. 60/210,733 entitled “Temperature-Controlled High Frequency Ablation for Creation of Transmyocardial Channels” to Dietz et al. filed Jun. 12, 2001, and incorporates the entirety of same by reference herein. This patent application is also a continuation-in-part of U.S. patent application Ser. No. 09/453,096 entitled “Medical Device and Method for Transmyocardial Revascularization” to Dietz et al. filed Dec. 2, 1999, which is a divisional of U.S. patent application Ser. No. 09/113,382, now abandoned, entitled “Medical Device and Method for Transmyocardial Revascularization” to Dietz et al. filed Jul. 10, 1998, and incorporates the respective entireties of same by reference herein.

### FIELD OF THE INVENTION

This invention is generally directed to the field of surgery, and more particularly to surgery procedures to improve the flow of blood to the heart muscle.

### BACKGROUND OF THE INVENTION

The number and variety of medical methods available to repair the effects of cardiovascular disease has increased rapidly over the last several years. More particularly, alternatives to open heart surgery and cardiovascular by-pass surgery have been extensively investigated, resulting in non-surgical procedures such as percutaneous transluminal coronary angioplasty, laser angioplasty, and atherectomy. These procedures are primarily directed toward the reduction of stenosis within the vasculature of a patient by either expanding the lumen through the use of a balloon, or ablating or otherwise removing the material making up the stenosis.

While such procedures have shown considerable promise, many patients still require bypass surgery due to such conditions as the presence of extremely diffuse stenotic lesions, the presence of total occlusions and the presence of stenotic lesions in extremely tortuous vessels. Also, some patients are too sick to successfully undergo bypass surgery, and because the above treatments require surgical backup in the case of complications, they are untreatable. Some patients requiring repeat bypass surgeries are also untreatable, such as those whose coronary arteries of insufficient internal diameter to serve as target vessels for bypass implantation; heart transplantation is regarded as the only therapeutic measure currently available for such patients.

One alternative to the foregoing procedures is known as Trans-Myocardial Revascularization (TMR). In TMR, channels are formed in the ventricle wall. Theoretically, such channels can provide blood flow directly from the left ventricular chamber to ischemic heart muscle. A history and description of this method is presented by Dr. M. Mirhoseini and M. Cayton in “Lasers in Cardiothoracic Surgery” in Lasers in General Surgery (Williams & Wilkins; 1989) pp. 216-223. To date, the most common clinical experimental approach to forming such channels employs a laser. See, for example, U.S. Pat. No. 5,755,714 to Murphy-Chutorian and U.S. Pat. No. 5,380,316 to Aita et al. Such channel forming attempts with laser technology, however, have largely been unsuccessful. In particular, most channels formed by laser means become occluded owing to the body's inflammatory response. As a result, the channels quickly become unavailable to deliver the blood to surrounding tissue.

Besides laser-based TMR, others have proposed mechanical means, the application of heat energy, or both to form channels in cardiac tissue. See, for example, European Patent Application EP 0 829 239 A1 to Mueller. To date, however, such attempts have been unsuccessful. In an abstract from the 70th Scientific Sessions of the American Heart Association (published in Circulation '96, No. 8, Suppl. I, pages 1-217), McKenna et al report the use of RF energy to form channels in cardiac tissue. The reported results are similar to those obtained using laser-based TMR, namely, that channels become occluded. McKenna et al did not report the use of temperature control for the delivered RF.

Still other TMR and TMR-like measures have been attempted to treat coronary heart disease (CHD) as alternatives to traditional bypass grafting and interventional coronary artery procedures. For example, some investigators have implanted various kinds of tubes into the myocardium having openings to the left ventricle. Transmyocardial puncturing was first described by White and Hishey as a method to restore blood supply in emergency situations. Sen et al. studied the possibility of transventricular needle puncture in acute ischemic myocardium. Walter et al. continued these studies by creating channels with cannulas of a diameter of 1.4 to 4.0 mm. Despite good initial success rates, long term patency has not been documented in any of these studies, however. More recently, transmyocardial holes created by laser ablation are described by Mirhoseini et al. In concordance with Mirhoseini and Horvath et al., where long term patency of channels is demonstrated. Sequential histologic examination of laser generated channels shows that an inflammatory process leads to fibrosis and consequent occlusion of channels, however.

Although laser transmyocardial revascularization (PMR) performed in patients having end stage CHD reduces symptoms in most patients, no improvement in myocardial function or long term patency of the channels has been documented. One of the major disadvantages of PMR procedures is that open heart surgery is required, which is accompanied by a mortality rate of up to 20%.

Patents and printed publications describing various aspects of the foregoing problems and the state of the art are listed below.

1. Beck, C. S.: The development of a new blood supply to the heart by operation. Ann Surg 1933; 102:801

2. Vineberg A. M., Walker J: Development of an anastomosis between the coronary vessels and a transplanted mammary artery. Can Med Assoc J 1964; 55: 117.

3. White M, Hershey J E: Multiple transmyocardial puncture revascularization in refractory ventricular fibrillation due to myocardial ischemia. Ann Thorac Surg 1968; 6: 557.

4. Sen P K, Udwadia T E, Kinare S G: Transmyocardial acupuncture. J Thorac Cardiovasc Surg 1965; 50:181.

5. Walter P, Hundeshagen H, Borst H G: Treatment of acute myocardial infarction by transmural blood supply from the ventricular cavity. Eur Surg Res 1971; 3: 130.

6. Mirhoseini M: Revascularization of the heart with laser. J Microvasc Surg 1981; 2: 253.

7. Mirhoseini M, Shelgikar S, Cayton M M: New concepts in revascularization of the myocardium. Ann Thorac Surg 1988, 45: 415-12.

8. Horvath K A, Smith W J, Laurence R G, et al: Recovery and viability of an acute myocardial infarct after transmyocardial laser revascularization. J Am Coll Cardiol 1995; 25: 258-63.

9. Wittkampf F H, Wever E F, Derksen R, et al: LocaLisa: new technique for real-time 3-dimensional localization of regular intracardiac electrodes. Circulation 1999; 99: 1312-1317.

Broadly, it is the object of the present invention to provide an improved method for performing TMR. It is a yet further object of the present invention to provide a method for performing TMR which results in channel formation which permits chronically increased blood circulation in the region of cardiac tissue adjacent to the channels.

All patents and printed publications listed hereinabove are hereby incorporated by reference herein, each in its respective entirety. As those of ordinary skill in the art will appreciate readily upon reviewing the drawings set forth herein and upon reading the Summary of the Invention, Detailed Description of the Preferred Embodiments and claims set forth below, at least some of the devices and methods disclosed in the patents and publications listed hereinabove may be modified advantageously in accordance with the teachings of the present invention.

### SUMMARY OF THE INVENTION

Various embodiments of the present invention have certain objects. That is, various embodiments of the present invention provide solutions to problems existing in the prior art, including, but not limited to, the problems listed above and problems such as: (a) intramyocardial channels created by laser energy tending to occlude over time; (b) necrotic zones forming around laser created channels having unpredictable extents and occurring at unpredictable frequencies; (c) channels in cardiac tissue formed by laser means are transmural, with the consequence that epicardial holes may result in pericardial bleeding; (d) not measuring the temperature of cardiac tissue adjacent the channels during PMR, with the consequence that cardiac tissue heating is controlled inadequately and channels of unpredictable morphology, necrotic zone extent and shape result; (e) cardiac tissue temperatures exceeding 100° C., resulting in tissue burning and charring, and subsequent inflammatory response; (f) local variations in cardiac tissue anatomy leading to structures such as adjoining blood vessels causing excessive convective heat loss or requiring highly variable energy levels; and (g) barotrauma being associated with laser-created channels.

Various embodiments of the present invention have certain advantages, including, without limitation, one or more of: (a) increasing the number of channels formed in cardiac tissue remaining open and unoccluded; (b) eliminating the occurrence of barotrauma since barotrauma is not associated with the delivery of radio-frequency energy to cardiac tissue; (c) stabilizing channels formed in cardiac tissue so that perfusion of cardiac tissue can continue for a significant period of time after the channels have been formed; (d) permitting a high degree of temperature control when forming channels in cardiac tissue; (e) avoiding the burning or charring of cardiac tissue during channel formation; (f) avoiding the formation of necrotic zones of excessive extent adjacent to the channels; and (g) minimizing the occurrence of epicardial holes.

Various embodiments of the present invention have certain features, including one or more of the following: (a) the formation of well defined and predictable necrotic zones adjacent to cardiac tissue channel; (b) a temperature sensor disposed near, on or in a cardiac tissue piercing and energy delivery means; (c) an extendable and retractable piercing means configured to pierce the myocardium to a preset depth; (d) temperature sensor means in combination with radio-frequency energy delivery means in a feedback control system resulting in the energy being delivered to cardiac tissue being automatically adapted to local anatomical variations in cardiac tissue.

The present invention relates to a transluminal medical system for revascularization of portions of a human heart through the formation of intramyocardial channels in cardiac tissue using piercing and radio-frequency energy delivery means. The channel forming system of the present invention may be configured in two basic embodiments: (a) a first embodiment where a transluminal steering and delivery system comprises a guide catheter or sheath which functions largely apart from and independent of other components in channel forming system (with the notable exception of a means for extending and retracting a piercing means from a distal end of the guide catheter or sheath), and (b) a second embodiment where a transluminal steering and delivery system is integrated into and combined with other components of the channel forming system, such as a piercing means, means for delivering radio-frequency energy to the piercing means, and temperature sensing means.

In one embodiment of the system of the present invention, there are provided a catheter delivered needle for piercing heart tissue, a radio-frequency energy source coupled to the needle, and means for controlling the radio-frequency energy source so that a predetermined temperature is obtained and not exceeded in the cardiac tissue adjacent the needle. The controller may also be configured to limit the amount time during which radio-frequency energy is delivered to the cardiac tissue. The needle may be cylindrical and have a piercing length of between about 3 mm and about 9 mm, and a piercing diameter of between about 0.5 mm and about 1.5 mm.

In a further embodiment of the system of the present invention, an ultrasonic sensor senses the distance from the distal end of the piercing means to an outer surface of the heart. Additionally, the piercing means may be configured to deliver a pharmacological agent to the cardiac tissue, such as physiologic mediators (e.g., growth factors, RNA, DNA or cDNA) or drugs for enhancing blood flow.

### BRIEF DESCRIPTION OF THE DRAWINGS

The present invention will become better understood by reference to the following Detailed Description of the Preferred Embodiments of the present invention when considered in connection with the accompanying Figures, in which like numbers designate like parts throughout, and where:

FIG. 1 is a view of a first embodiment of the present invention configured to access the endocardial surface of the left ventricle.

FIG. 2 depicts the distal tip 10 of catheter 1 of FIG. 1.

FIG. 3 depicts a second embodiment of the present invention.

FIG. 4 shows an end view of the embodiment shown in FIG. 3.

FIG. 5 shows a third embodiment of the present invention.

FIG. 6 depicts one method of the present invention.

FIG. 7 depicts a further embodiment of the present invention.

FIG. 8 depicts another embodiment of the present invention.

FIG. 9 depicts yet a further embodiment of the present invention.

### DETAILED DESCRIPTION OF THE PREFERRED EMBODIMENTS

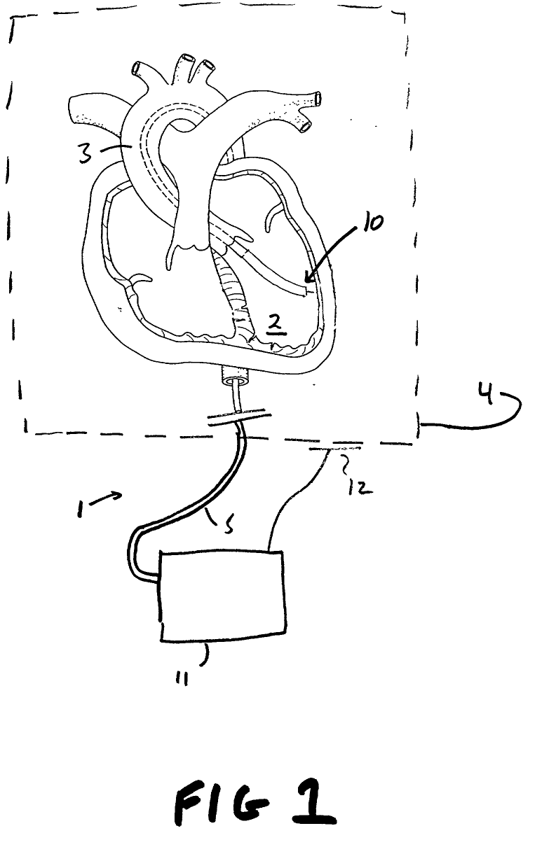

FIG. 1 shows a first embodiment of the present invention disposed within a human body, and is configured to access the endocardium of the left ventricle. Catheter 1 is introduced into the left ventricle via aorta 3 using well known femoral percutaneous access methods. Although such an access method is preferred in some embodiments of the present invention, other access methods may also be employed, such as a direct surgical cut down which exposes an exterior or interior portion of the heart requiring revacularization. Catheter 1 comprises catheter body 5 and distal tip 10. Catheter body 5 typically comprises a biocompatible polymeric sheath, and has one or more conductors or guide lumens disposed therewithin. The particular design of catheter body 5 depends on the design of the distal tip 10, as well as the particular handling characteristics an individual physician prefers. For example, the catheter body may be made to be more or less stiff along its entire length or along various portions of its length, as is well known in the art. Moreover, the catheter body may include one or more guide lumens, or one or more guide catheters, or both. Still further, the catheter body may feature rapid exchange capabilities to permit a guide wire to be introduced first into a patient's body, followed by the guide catheter being advanced to a site within the patient's body over the wire.

The proximal end of the catheter body couples to a radio-frequency (RF) energy generator 11 that delivers “Radio Frequency Energy (RF Energy)”. In the illustrated embodiment, the system operates in a monopolar mode. In this mode the system requires skin patch electrode 12 that serves as an indifferent second electrode, as is well known in the art. In an alternative embodiment the system could also be operated in a bipolar mode in which catheter 1 includes two electrodes.

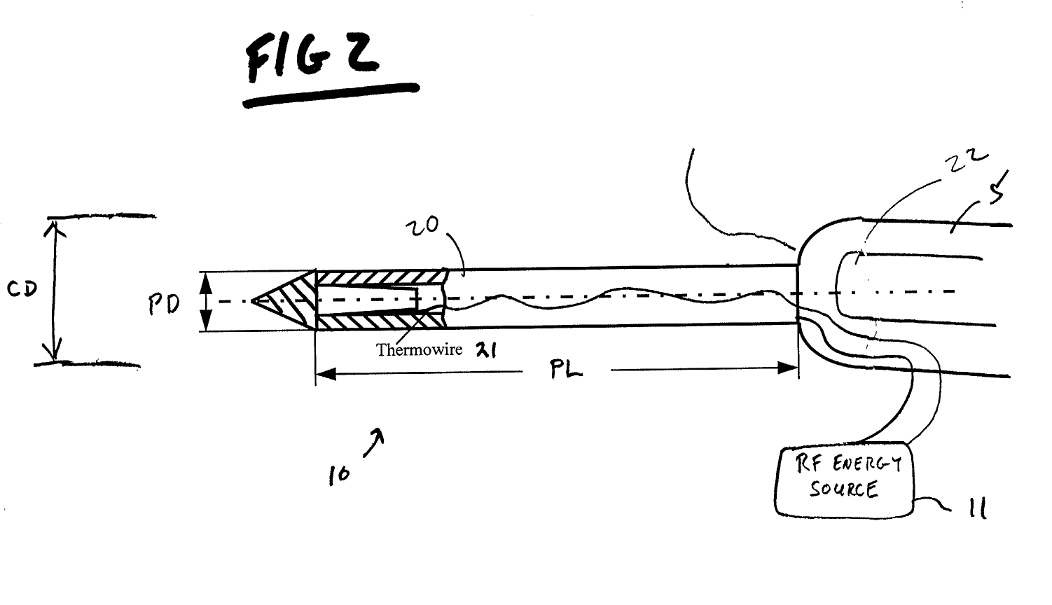

FIG. 2 depicts the distal tip 10 of catheter 1. As seen, distal tip 10 essentially comprises piercing means or needle 20 disposed distally from the distal end of the catheter body 5. Needle 20 is preferably cylindrically shaped and has a sharpened tip. Needle 20 may have a piercing length PL of between about 3 mm and about 9 mm, with 7 mm being a preferred PL. A maximum piercing diameter PD of between about 0.5 mm and about 1.5 mm for needle 20 is preferred, with 0.7 mm being most preferred. Needle 20 may cooperate with the catheter body, and in particular with the catheter diameter “CD” of the catheter body, such that the piercing length of the needle cannot exceed the total needle length. If the distal end of the catheter body is appropriately configured, shaped and sized it will not follow the needle through the heart when certain ranges of force are applied. In one preferred embodiment of the present invention, the catheter body is at least 3 French in diameter. Of course, the particular dimensions of the catheter body depend on the patient's condition and a physician's preference. Thus, piercing means and catheter having greater or lesser lengths or diameters may also be used. Moreover, while the needle is shown as made of metal, the needle and/or various portions of the needle need not be formed of metal. For example, the end cap may be formed of glass or polymer. The needle may also be provided without any end cap.

Needle 20 includes within or near it a temperature sensing device, here illustrated as a thermowire 21. Thermowire 21 permits the temperature of the tissue in the region of the needle to be reliably measured. Thermowire 21 is coupled to RF energy generator 11. Needle 20 is also coupled to RF energy generator 11. Also illustrated is guide lumen 22 disposed within an interior portion of catheter body 5. Guide lumen 22 may be provided to permit a stylet or other control devices to be inserted within catheter body 5 and thereby permit more precise control of needle 20. The particular design of catheter body 5 may be selected from among many known designs.

FIG. 3 depicts another embodiment of the present invention. Multiple piercing needles 35-1 and 35-2 are disposed distally from the distal end of catheter body 5. Each of needles is preferably cylindrically shaped and has a thermosensor disposed therein or nearby.

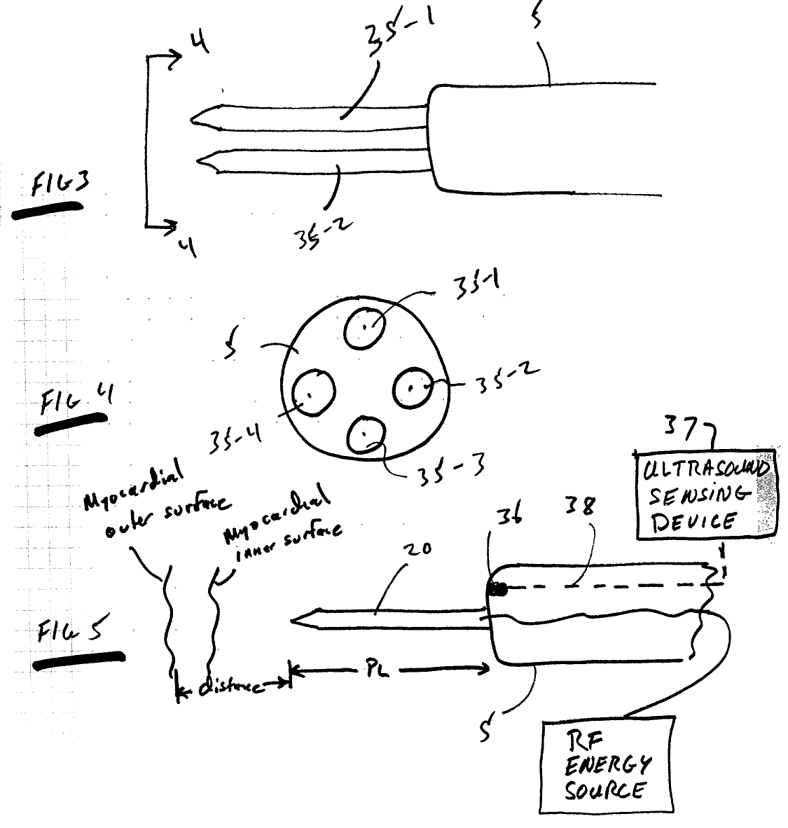

FIG. 4 shows an end view of the embodiment shown in FIG. 3 Several needles 35-1, 35-2, 35-3 and 35-4 are disposed distally from catheter body 5. In this embodiment, multiple channels may be created during a single piercing procedure. While four needles are illustrated, more or less needles may also be employed.

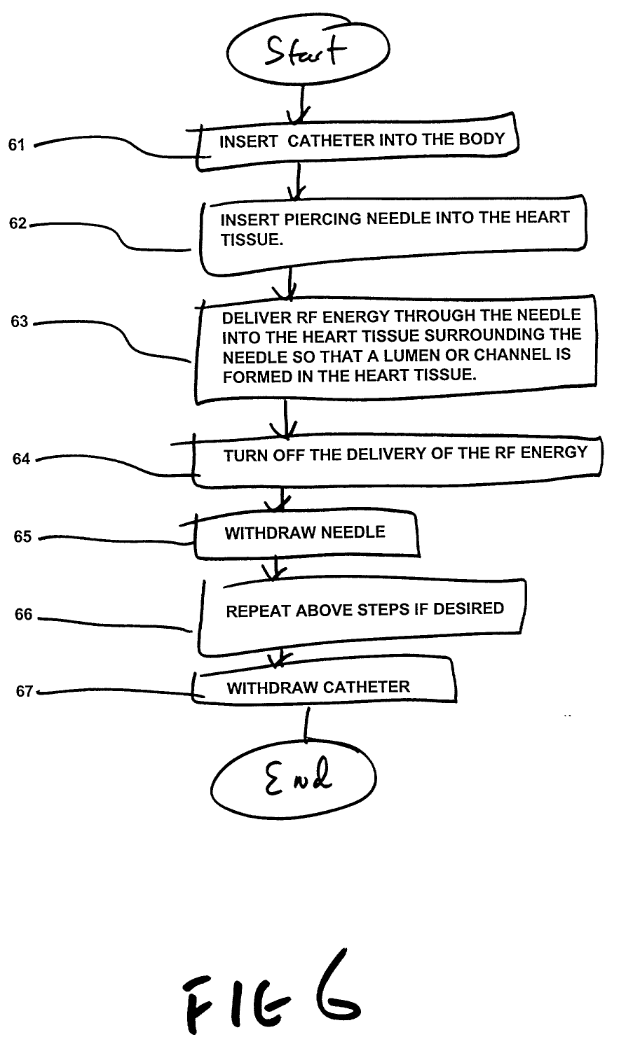

FIG. 5 shows a further embodiment of the present invention. The distal end of catheter body 5 features a piercing needle 20. Catheter body 5 and piercing needle 20 have a design similar to those shown above in FIG. 2, but further comprise ultrasonic sensor 36 disposed at or near at the distal end of catheter body 5. Ultrasonic sensor 36 is coupled to ultrasonic sensing driver or device 37 through conductor(s) 38. Ultrasonic sensor 36 and driver 37 may be configured in any suitable manner, including those disclosed in Patents WO 9517131, WO 9526677, EP 740565, EP 739183, WO 9519201, WO 9515784, EP 474957 to Ferek-Petric. Sensor 36 and driver 37 permit the distance between catheter distal end 36 and the surface of the cardiac tissue to be measured. Such surfaces include both inner as well as outer myocardial surfaces when the catheter is used in an endocardial manner. Such sensors therefore permit needle 20 to be introduced into the myocardium, but without piercing therethrough into the pericardial space. As is well known in the art, perforation of the myocardium permits bleeding of the heart to occur from inside out. Such bleeding is known as pericardial effusion and may lead to serious complications. Ultrasonic sensing driver 37 may further include a controller to permit the distance of the sensed heart surface to be correlated with the length of piercing needle 20 such that the distance from the sharpened tip of the piercing needle to the finer outer surface of the local region of the myocardium may be calculated and displayed to the physician.

FIG. 6 depicts one method of the present invention. At step 61 a catheter having one or more piercing needles as described above is inserted into the body. At step 62 the piercing needle or needles is inserted into the heart tissue. At step 63 RF energy is delivered through the needle into the heart tissue surrounding the needle so that a lumen or channel is formed in the heart tissue. This step may further include the step of modulating the RF energy delivered in accordance with the temperature sensed at or near needle 20 so that the temperature of cardiac tissue surrounding needle 20 does not exceed a predetermined temperature. This step may also include delivering the modulated RF energy for a predetermined period of time between approximately 5-25 seconds with 15 seconds preferred. Of course, the particular period of time over which energy is delivered depends on the patient's physiology, the doctor's preferences, the piercing needle's size and geometry, the temperature set points employed and other factors. In a preferred embodiment, the temperature set point ranges between about 60° C. and about 80° C., with 70° C. being preferred. The temperature set point is important because the highest temperature reached in the cardiac tissue surrounding the needle determines the amount and degree of necrosis that will form. The inventors believe that the temperature ranges set forth herein minimize the ultimate zone of necrosis. In step 64 RF energy is turned off and in step 65 the needle is withdrawn. At step 66 the procedure may be repeated at another location. Finally, the catheter is withdrawn at step 67.

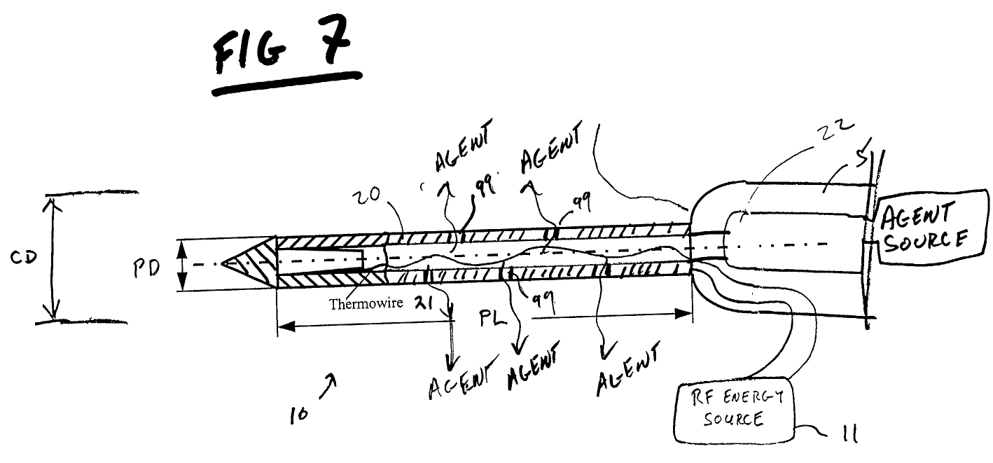

Step 64 may further include delivering a pharmacological agent through the needle while it is still inserted in the heart tissue. A catheter suitable for such delivery is shown in FIG. 7. Such pharmacological agents may include vasodilators, anticoagulants, platelet inhibitors, growth factors stimulating angiogenesis or myocyte growth or their respective RNA, cDNA or DNA sequences.

It is also to be understood that step 62 may include providing a catheter, sensing the distance from the catheter's distal end to one or more surfaces of the heart, and calculating and displaying the distance between the sharpened distal tip of the needle to the sensed surface of the heart. In such a manner a physician may reliably control the depth to which needle 20 pierces cardiac tissue. The system of the present invention may also include a device for providing a “stop forward movement signal” to the physician to prevent transmural myocardial piercing.

FIG. 7 depicts a further embodiment of the present invention substantially the same as that shown in FIG. 2, but where agent infusion holes 99 in fluid communication with an agent source. As discussed above in FIG. 6, the system and of the present invention may deliver one or more pharmacological agents through needle 20 while inserted in cardiac tissue. Such pharmacological agents may include vasodilators, anticoagulants, platelet inhibitors, growth factors stimulating angiogenesis or myocyte growth or their respective RNA, cDNA or DNA sequences.

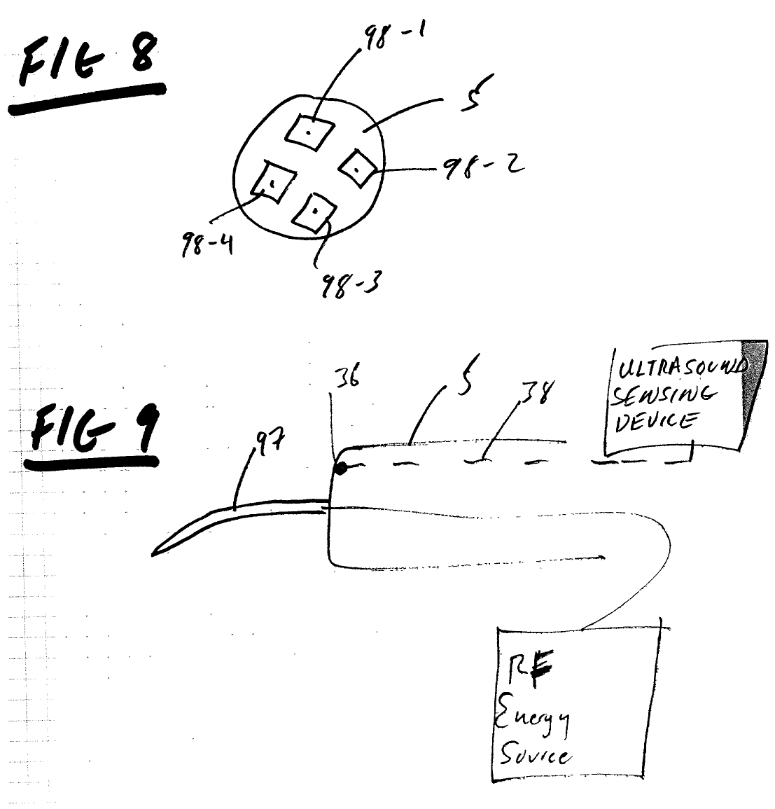

FIG. 8 depicts a further embodiment of the present invention substantially the same as that shown in FIG. 4, but where needles 98-1 through 98-4 are square in cross-section.

FIG. 9 depicts a further embodiment of the present invention substantially the same as that shown in FIG. 5, but where needle 97 is curved along its length and in a single plane. Of course other types of curves may be used, including multi-planar curves, helixes, and so on.

### EXAMPLE 1

Transmyocardial channels were created using a radiofrequency (RF) probe. The catheter consisted of a 4 F application catheter having a cylindrical ablation electrode (Ø 0.8 and 1 mm, 5 mm long) and a sharpened conus. A thermocouple was incorporated in the center of the ablation electrode. We evaluated the impact of temperature- and power controlled applications on the resulting channel dimensions and shape, and the size of surrounding necrosis. In 12 anesthetized rabbits the RF probe (Ø 0.8 mm )was introduced from the epicardial surface via a thoracotomy for 4-7 applications along the left ventricular (LV) wall. Transmyocardial channels were created by either temperature controlled (in 5 rabbits) or power controlled (in 7 rabbits) applications for 3-10 seconds. The RF probe was then removed. The experiments were terminated after 4 h. The dimensions of transmyocardial channels and zones of necrosis were measured using an automatic morphometric system and cross sections stained with HE and Fuchsin, respectively. By this, the mean diameter of transmyocardial channels and necrosis was calculated. The shape of the transmyocardial channels was analyzed using HE stained cross sectional slices. Persistent transmyocardial channels could be identified in 22/25 of the temperature controlled applications and in 28/35 of the power controlled applications. Temperature- and power controlled applications yield in transmyocardial channels with diameters ranging from 113 to 743 μm. The channels had a more round shape created with temperature control as compared to power control with a comparable maximum temperature. The diameters of the channels created with power controlled energy delivery correlated poorly with the duration (r=0.1), the energy-time product (r=0.08), and the max. temperature (r=0.1). Diameters of transmyocardial channels created with temperature controlled energy delivery were weakly correlated with duration (r=0.4) and the ablation temperature (r=0.35), but highly correlated with the temperature-time product (r=0.8). The diameter of the necrosis was correlated with the max. temperature in both groups, r=0.9 for temperature control and r=0.58 for power control, respectively, but not correlated with the temperature-time product in the temperature controlled group (r=0.3).

The creation of persistent channels was discovered to depend on the temperature time product of the RF energy administered. Thus, a temperature-controlled energy delivery is necessary for the reproducible generation of transmyocardial channels that remain detectable for at least 4 hours.

### EXAMPLE 2

A catheter system comprising an 8 F guiding catheter in which a 6 F guiding catheter was used together with a 4 F application catheter. A cylindrical ablation electrode 1 mm in diameter and 5 mm in length with a sharpened conus was employed. A thermocouple was incorporated into the center of the ablation electrode to permit temperature-controlled energy delivery. At the end of the series of these experiments a 7 F catheter with an extractable needle and bi-directional steerability was employed. In 9 anesthetized pigs (2535 kg) the system was introduced by a transfemoral approach. The ablation electrode was inserted into the myocardium for its entire length followed by temperature controlled HF energy delivery with a target temperature of 75° C. for 5 s. Fifteen piercings without energy application were performed in one pig. The LETR principle was used in 5 animals to assess the contact and introduction of the needle electrode into the myocardium. The LETR-principle is based on the hypothesis that the temperature rise resulting from the application of low levels of radiofrequency energy (0.1W) varies according to the amount or degree of electrode-tissue contact. A firm electrode-tissue contact causes a relatively high temperature increase, whereas a poor contact causes a low temperature increase.

A LocaLisa system was used in two animals to determine the spatial position of the needle electrode and to mark the location of the channels. The LocaLisa system features an orthogonal lead configuration where three independent alternating currents of 1 mA each are delivered through the patient's chest, with frequencies of 30.27 kHz, 30.70 kHz, and 31.15 kHz being employed for the transversal, axial, and saggital directions, respectively. The LocaLisa system has two input amplifiers for measuring the resulting sensed signals on two mapping catheter electrodes relative to a stable skin or catheter reference electrode. The amplitudes of the three frequency components were optically transmitted to a Macintosh computer. A custom-designed software application provided moving-average filtering, calibration, and real-time display of the position of the distal portion of the mapping catheter. During energy delivery temperature, RF power and impedance were continuously recorded with a computer.

Six pigs were harvested one hour after the procedure (acute pigs) and three after 3 weeks (chronic pigs). Histological examination was done in serial sections of 5 μm thickness stained with H&E and fuchsin in the acute pigs, and with Elastica v. Gieson in the chronic pigs. The ferret diameter of the channels, the necrotic zone and the fibrotic zone were calculated. The shapes of the channels and the degrees of obstruction were assessed.

It was determined that a total of 107 channels were stabilized using radio-frequency energy delivery and an additional 15 channels were stabilized without energy delivery. In the 107 cases, the average temperature achieved was T_{avg}=70.4±2.7° C. (61-76° C.) requiring an average RF power P_{avg}=3.9±4.2W (1-30W). The impedance between the RF probe and the indifferent electrode averaged Imp=171±32 Ω (104-242 Ω). Hemodynamic parameters remained stable in all but one animal which died because of ventricular fibrillation. Three pigs had minor pericardial effusions. The endocardial ostium could be identified in 88 of the 107 cases. Histomorphometry of the channels and the necrotic zone was done in 46 out of 67 cases for acute pigs, and of the ferrit diameter in 33 out of 39 cases for chronic pigs. 65% of the acute pigs had channels having oval shapes. 85% of the acute pigs had patent channels. The mean degree of obstruction was 50%, where obstructive material consisted of thrombus. In the acute pigs the ferret diameter of the channels was 850±456 μm and that of the necrotic zone 3100±700 μm. In the chronic pigs 2 patent channels were found as well as 31 channel remnants containing a partially recanalized thrombus surrounded by a dense mesh of capillaries. The ferret diameter of the fibrotic zone was 2800±850 μm.

PMR was shown to be feasible using radio-frequency TMR. Reproducible intramyocardial channels were created that persisted and stayed open for at least 1 hour in a high percentage of cases. After 3 weeks, intense neovascularization of the fibrotic zone was observed.

Although specific embodiments of the invention are described here in some detail, it is to be understood that those specific embodiments are presented for the purpose of illustration, and are not to be taken as somehow limiting the scope of the invention defined in the appended claims to those specific embodiments. It is also to be understood that various alterations, substitutions, and modifications may be made to the particular embodiments of the present invention described herein without departing from the spirit and scope of the appended claims.

In the claims, means plus function clauses are intended to cover the structures and devices described herein as performing the recited function and their equivalents. Means plus function clauses in the claims are not intended to be limited to structural equivalents only, but are also intended to include structures and devices which function equivalently in the environment of the claimed combination.

All printed publications, patents and patent applications referenced hereinabove are hereby incorporated by referenced herein, each in its respective entirety.

We claim:

1. A transluminal intramyocardial channel forming system for creating intramyocardial channels in cardiac tissue of a patient's heart, comprising:
(a) means for forming at least one intramyocardial channel in cardiac tissue, the intramyocardial channel forming means comprising a first distal end and a second proximal end, comprising:
(i) first means for piercing cardiac tissue having a sharpened distal tip, a piercing length of between about 3 mm and about 9 mm, and a maximum piercing diameter of between about 0.5 mm and about 1.5 mm, the piercing means being disposed adjacent to the first distal end;
(ii) means for delivering radio-frequency energy to the piercing means, the radio-frequency energy delivering means comprising means for generating and controlling the amount of radio-frequency power delivered to the piercing means, the radio-frequency energy delivering means being disposed adjacent to the second proximal end and being operably connected to the piercing means;
(iii) means, adjacent to the piercing means, for sensing and relaying a feedback control signal indicative of a cardiac tissue temperature, the temperature sensing means being operably connected to the radio-frequency energy delivering means to permit the feedback control signal to be relayed thereto; and
(iv) an electrode adjacent to the piercing means and operably connected to the radio-frequency energy delivering means;
wherein the radio-frequency energy delivering means, the radio-frequency power controlling means and the temperature sensing means are interconnected and configured to form an integrated feedback control system, the feedback control system being configured to maintain the cardiac tissue temperature between about 50 degrees Centigrade and about 100 degrees Centigrade for a period of time ranging between about 1 seconds and about 50 seconds when the distal tip of the piercing means is disposed in cardiac tissue to form an intramural channel therein, and further wherein the feedback control system is configured to form necrotic zones of minimum thickness in the channel, and
(b) means for transluminally delivering the piercing means to the cardiac tissue comprising at least one of a lumen and an over-the-wire means for accepting at least portions of the intramyocardial channel forming means therewithin, thereon or therethrough, the transluminal delivery means further comprising:
(i) a third distal end, and
(ii) a fourth proximal end.

2. The transluminal intramyocardial channel forming system of claim 1, wherein the piercing means further comprises a metal cylinder having a pointed hollow tip.

3. The transluminal intramyocardial channel forming system of claim 1, further comprising means for sensing a distance between the distal tip and an outer surface of the cardiac tissue.

4. The transluminal intramyocardial channel forming system of claim 3, wherein the distance sensing means comprises means for ultrasonically sensing the outer surface of the cardiac tissue.

5. The transluminal intramyocardial channel forming system of claim 3, wherein the distance sensing means is disposed proximally from the piercing means.

6. The transluminal intramyocardial channel forming system of claim 1, further comprising means for delivering a pharmacological agent through at least one of the first distal end, the third distal end and the piercing means to the cardiac tissue.

7. The transluminal intramyocardial channel forming system of claim 1, further comprising a second means for piercing cardiac tissue having a sharpened distal tip.

8. The transluminal intramyocardial channel forming system of claim 1, wherein the piercing means comprises at least one needle having one of a rectangular and a square cross-section.

9. The transluminal intramyocardial channel forming system of claim 1, wherein the piercing means comprises at least one curved needle.

10. The transluminal intramyocardial channel forming system of claim 1, wherein at least portions of the transluminal delivery means form a delivery catheter having an outer diameter ranging between about 4 French and about 12 French.

11. The transluminal intramyocardial channel forming system of claim 1, wherein at least portions of the transluminal delivery means form a delivery catheter having an outer diameter ranging between about 5 French and about 10 French.

12. The transluminal intramyocardial channel forming system of claim 1, wherein at least portions of the transluminal delivery means form a delivery catheter having an outer diameter ranging between about 6 French and about 8 French.

13. The transluminal intramyocardial channel forming system of claim 1, wherein the radio-frequency energy generating and controlling means is configured to deliver between about 0.5 Watts and about 50 watts to the piercing means.

14. The transluminal intramyocardial channel forming system of claim 1, wherein the radio-frequency energy generating and controlling means is configured to deliver between about 1 Watt and about 30 watts to the piercing means.

15. The transluminal intramyocardial channel forming system of claim 1, wherein the radio-frequency energy generating and controlling means is configured to deliver between about 2 Watts and about 25 watts to the piercing means.

16. The transluminal intramyocardial channel forming system of claim 1, wherein the radio-frequency energy generating and controlling means is configured to deliver between about 3 Watts and about 20 watts to the piercing means.

16. The transluminal intramyocardial channel forming system of claim 1, wherein an impedance measured between the piercing means and the electrode ranges between about 10 Ohms and about 500 Ohms.

17. The transluminal intramyocardial channel forming system of claim 1, wherein an impedance measured between the piercing means and the electrode ranges between about 50 Ohms and about 350 Ohms.

18. The transluminal intramyocardial channel forming system of claim 1, wherein an impedance measured between the piercing means and the electrode ranges between about 100 Ohms and about 250 Ohms.

19. The transluminal intramyocardial channel forming system of claim 1, further comprising means for determining a spatial position of the distal tip of the piercing means in the patient's heart.

20. The transluminal intramyocardial channel forming system of claim 19, wherein the spatial position determining means is selected from the group consisting of an X-Ray imaging system, an ultrasonic imaging system, an orthogonal magnetic field sensing system, an alternating current orthogonal electromagnetic field sensing system, and a fluoroscopic system.

21. The transluminal intramyocardial channel forming system of claim 1, wherein the the feedback control system is configured to maintain the cardiac tissue temperature between about 55 degrees Centigrade and about 90 degrees Centigrade when the distal tip of the piercing means is disposed in cardiac tissue to form the intramural channel therein.

22. The transluminal intramyocardial channel forming system of claim 1, wherein the the feedback control system is configured to maintain the cardiac tissue temperature between about 60 degrees Centigrade and about 80 degrees Centigrade when the distal tip of the piercing means is disposed in cardiac tissue to form the intramural channel therein.

23. The transluminal intramyocardial channel forming system of claim 1, wherein the feedback control system is configured to maintain the cardiac tissue temperature between about 65 degrees Centigrade and about 75 degrees Centigrade when the distal tip of the piercing means is disposed in cardiac tissue to form the intramural channel therein.

24. The transluminal intramyocardial channel forming system of claim 1, wherein the feedback control system is configured to maintain the cardiac tissue temperature for a period of time ranging between about 2 seconds and about 45 seconds when the distal tip of the piercing means is disposed in cardiac tissue to form the intramural channel therein.

25. The transluminal intramyocardial channel forming system of claim 1, wherein the feedback control system is configured to maintain the cardiac tissue temperature for a period of time ranging between about 2 seconds and about 30 seconds when the distal tip of the piercing means is disposed in cardiac tissue to form the intramural channel therein.

26. The transluminal intramyocardial channel forming system of claim 1, wherein the feedback control system is configured to maintain the cardiac tissue temperature for a period of time ranging between about 5 seconds and about 25 seconds when the distal tip of the piercing means is disposed in cardiac tissue to form the intramural channel therein.

27. The transluminal intramyocardial channel forming system of claim 1, wherein the transluminal delivery means further comprises a steerable guiding catheter.

28. The transluminal intramyocardial channel forming system of claim 1, wherein the transluminal delivery means further comprises a non-steerable guiding sheath comprising a distal end, the distal end having a pre-shaped curve.

29. The transluminal intramyocardial channel forming system of claim 1, wherein the transluminal delivery means further comprises a handle disposed adjacent the fourth proximal end.

30. The transluminal intramyocardial channel forming system of claim 29, wherein the transluminal delivery means further comprises means, operably connected to the handle, for at least bi-directionally steering the third distal end transluminally to the cardiac tissue.

31. The transluminal intramyocardial channel forming system of claim 1, further comprising means for controllably retracting the piercing means inside a protective distal housing or sheath.

32. The transluminal intramyocardial channel forming system of claim 1, further comprising means for controllably extending the piercing means distally beyond a protective distal housing or sheath.

33. The transluminal intramyocardial channel forming system of claim 1, wherein the temperature sensing means is a thermosensor.

34. A transluminal intramyocardial channel forming system for creating intramyocardial channels in cardiac tissue of a patient's heart, comprising:
(a) first means for piercing cardiac tissue having a sharpened distal tip, a piercing length of between about 3 mm and about 9 mm, and a maximum piercing diameter of between about 0.5 mm and about 1.5 mm, the piercing means being disposed adjacent to the first distal end;
(b) means for delivering radio-frequency energy to the piercing means, the radio-frequency energy delivering means comprising means for generating and controlling the amount of radio-frequency power delivered to the piercing means, the radio-frequency energy delivering means being disposed adjacent to the second proximal end and being operably connected to the piercing means;
(c) means, adjacent to the piercing means, for sensing and relaying a feedback control signal indicative of a cardiac tissue temperature, the temperature sensing means being operably connected to the radio-frequency energy delivering means to permit the feedback control signal to be relayed thereto;
(d) an electrode adjacent to the piercing means and operably connected to the radio-frequency energy delivering means, and
(e) means for steering and delivering the piercing means transluminally to the cardiac tissue;
wherein the radio-frequency energy delivering means, the radio-frequency power controlling means and the temperature sensing means are interconnected and configured to form an integrated feedback control system, the feedback control system being configured to maintain the cardiac tissue temperature between about 50 degrees Centigrade and about 100 degrees Centigrade for a period of time ranging between about 1 seconds and about 50 seconds when the distal tip of the piercing means is disposed in cardiac tissue to form an intramural channel therein, and further wherein the feedback control system is configured to form necrotic zones of minimum thickness in the channel.

35. The transluminal intramyocardial channel forming system of claim 34, wherein the piercing means further comprises a metal cylinder having a pointed hollow tip.

36. The transluminal intramyocardial channel forming system of claim 34, further comprising means for sensing a distance between the distal tip and an outer surface of the cardiac tissue.

37. The transluminal intramyocardial channel forming system of claim 36, wherein the distance sensing means comprises means for ultrasonically sensing the outer surface of the cardiac tissue.

38. The transluminal intramyocardial channel forming system of claim 36, wherein the distance sensing means is disposed proximally from the piercing means.

39. The transluminal intramyocardial channel forming system of claim 34, further comprising means for delivering a pharmacological agent through at least one of the first distal end, the third distal end and the piercing means to the cardiac tissue.

40. The transluminal intramyocardial channel forming system of claim 34, further comprising a second means for piercing cardiac tissue having a sharpened distal tip.

41. The transluminal intramyocardial channel forming system of claim 34, wherein the piercing means comprises at least one needle having one of a rectangular and a square cross-section.

42. The transluminal intramyocardial channel forming system of claim 34, wherein the piercing means comprises at least one curved needle.

43. The transluminal intramyocardial channel forming system of claim 34, wherein at least portions of the steering and delivery means have an outer diameter ranging between about 4 French and about 12 French.

44. The transluminal intramyocardial channel forming system of claim 34, wherein at least portions of the steering and delivery means have an outer diameter ranging between about 5 French and about 10 French.

45. The transluminal intramyocardial channel forming system of claim 34, wherein at least portions of the steering and delivery means have an outer diameter ranging between about 6 French and about 8 French.

46. The transluminal intramyocardial channel forming system of claim 34, wherein the radio-frequency energy generating and controlling means is configured to deliver between about 0.5 Watts and about 50 watts to the piercing means.

47. The transluminal intramyocardial channel forming system of claim 34, wherein the radio-frequeny energy generating and controlling means is configured to deliver between about 5 Watt and about 30 watts to the piercing means.

48. The transluminal intramyocardial channel forming system of claim 34, wherein the radio-frequency energy generating and controlling means is configured to deliver between about 2 Watts and about 25 watts to the piercing means.

49. The transluminal intramyocardial channel forming system of claim 34, wherein the radio-frequency energy generating and controlling means is configured to deliver between about 3 Watts and about 20 watts to the piercing means.

50. The transluminal intramyocardial channel forming system of claim 34, wherein an impedance measured between the piercing means and the electrode ranges between about 10 Ohms and about 500 Ohms.

51. The transluminal intramyocardial channel forming system of claim 34, wherein an impedance measured between the piercing means and the electrode ranges between about 50 Ohms and about 350 Ohms.

52. The transluminal intramyocardial channel forming system of claim 34, wherein an impedance measured between the piercing means and the electrode ranges between about 100 Ohms and about 250 Ohms.

53. The transluminal intramyocardial channel forming system of claim 34, further comprising means for determining a spatial position of the distal tip of the piercing means in the patient's heart.

54. The transluminal intramyocardial channel forming system of claim 53, wherein the spatial position determining means is selected from the group consisting of an X-Ray imaging system, an ultrasonic imaging system, an orthogonal magnetic field sensing system, an alternating current orthogonal electromagnetic field sensing system, and a fluoroscopic system.

55. The transluminal intramyocardial channel forming system of claim 34, wherein the the feedback control system is configured to maintain the cardiac tissue temperature between about 55 degrees Centigrade and about 90 degrees Centigrade when the distal tip of the piercing means is disposed in cardiac tissue to form the intramural channel therein.

56. The transluminal intramyocardial channel forming system of claim 34, wherein the the feedback control system is configured to maintain the cardiac tissue temperature between about 60 degrees Centigrade and about 80 degrees Centigrade when the distal tip of the piercing means is disposed in cardiac tissue to form the intramural channel therein.

57. The transluminal intramyocardial channel forming system of claim 34, wherein the feedback control system is configured to maintain the cardiac tissue temperature between about 65 degrees Centigrade and about 75 degrees Centigrade when the distal tip of the piercing means is disposed in cardiac tissue to form the intramural channel therein.

58. The transluminal intramyocardial channel forming system of claim 34, wherein the feedback control system is configured to maintain the cardiac tissue temperature for a period of time ranging between about 2 seconds and about 45 seconds when the distal tip of the piercing means is disposed in cardiac tissue to form the intramural channel therein.

59. The transluminal intramyocardial channel forming system of claim 34, wherein the feedback control system is configured to maintain the cardiac tissue temperature for a period of time ranging between about 2 seconds and about 30 seconds when the distal tip of the piercing means is disposed in cardiac tissue to form the intramural channel therein.

60. The transluminal intramyocardial channel forming system of claim 34, wherein the feedback control system is configured to maintain the cardiac tissue temperature for a period of time ranging between about 5 seconds and about 25 seconds when the distal tip of the piercing means is disposed in cardiac tissue to form the intramural channel therein.

61. The transluminal intramyocardial channel forming system of claim 34, wherein the steering and delivery means further comprises a handle.

62. The transluminal intramyocardial channel forming system of claim 34, further comprising means for controllably retracting the piercing means inside a protective distal housing or sheath.

63. The transluminal intramyocardial channel forming system of claim 34, further comprising means for controllably extending the piercing means distally beyond a protective distal housing or sheath.

64. The transluminal intramyocardial channel forming system of claim 34, wherein the temperature sensing means is a thermosensor.

65. A transluminal method of forming intramyocardial channels in cardiac tissue of a patient's heart, the method employing a transluminal intramyocardial channel forming system comprising means for forming at least one intramyocardial channel in cardiac tissue, the intramyocardial channel forming means comprising a first distal end and a second proximal end, first means for piercing cardiac tissue having a sharpened distal tip, a piercing length of between about 3 mm and about 9 mm, and a maximum piercing diameter of between about 0.5 mm and about 1.5 mm, the piercing means being disposed adjacent to the first distal end, means for delivering radio-frequency energy to the piercing means, the radio-frequency energy delivering means comprising means for generating and controlling the amount of radio-frequency power delivered to the piercing means, the radio-frequency energy delivering means being disposed adjacent to the second proximal end and being operably connected to the piercing means, means, adjacent to the piercing means, for sensing and relaying a feedback control signal indicative of a cardiac tissue temperature, the temperature sensing means being operably connected to the radio-frequency energy delivering means to permit the feedback control signal to be relayed thereto, and an electrode adjacent to the piercing means and operably connected to the radio-frequency energy delivering means, wherein the radio-frequency energy delivering means, the radio-frequency power controlling means and the temperature sensing means are interconnected and configured to form an integrated feedback control system, the feedback control system being configured to maintain the cardiac tissue temperature between about 60 degrees Centigrade and about 80 degrees Centigrade for a period of time ranging between about 1 second and about 50 seconds when the distal tip of the piercing means is disposed in cardiac tissue to form an intramural channel therein, and further wherein the feedback control system is configured to form necrotic zones of minimum thickness in the channel, and means for transluminally delivering the piercing means to the cardiac tissue comprising at least one of a lumen and an over-the-wire means for accepting at least portions of the intramyocardial channel forming means therewithin, thereon or therethrough, the transluminal delivery means further comprising a third distal end, a fourth proximal end, a handle disposed adjacent the fourth proximal end, means, operably connected to the handle, for at least bi-directionally steering the third distal end transluminally to the cardiac tissue, the method comprising:
inserting the third distal end into a blood vessel of the patient which provides venous access to the patient's heart;
guiding the third distal end to the cardiac tissue;
piercing the cardiac tissue using the piercing means, and delivering radio-frequency energy to the piercing means to form the transmural channel.

66. A transluminal method of forming intramyocardial channels in cardiac tissue of a patient's heart, the method employing a transluminal intramyocardial channel forming system for creating intramyocardial channels in cardiac tissue of a patient's heart, the system comprising first means for piercing cardiac tissue having a sharpened distal tip, a piercing length of between about 3 mm and about 9 mm, and a maximum piercing diameter of between about 0.5 mm and about 1.5 mm, the piercing means being disposed adjacent to the first distal end, means for delivering radio-frequency energy to the piercing means, the radio-frequency energy delivering means comprising means for generating and controlling the amount of radio-frequency power delivered to the piercing means, the radio-frequency energy delivering means being disposed adjacent to the second proximal end and being operably connected to the piercing means, means, adjacent to the piercing means, for sensing and relaying a feedback control signal indicative of a cardiac tissue temperature, the temperature sensing means being operably connected to the radio-frequency energy delivering means to permit the feedback control signal to be relayed thereto, an electrode adjacent to the piercing means and operably connected to the radio-frequency energy delivering means, and means for steering and delivering the piercing means transluminally to the cardiac tissue having proximal and distal ends, wherein the radio-frequency energy delivering means, the radio-frequency power controlling means and the temperature sensing means are interconnected and configured to form an integrated feedback control system, the feedback control system being configured to maintain the cardiac tissue temperature between about 50 degrees Centigrade and about 100 degrees Centigrade for a period of time ranging between about 1 seconds and about 50 seconds when the distal tip of the piercing means is disposed in cardiac tissue to form an intramural channel therein, and further wherein the feedback control system is configured to form necrotic zones of minimum thickness in the channel, the method comprising:
inserting the distal end of the guiding and steering system into a blood vessel of the patient which provides venous access to the patient's heart;
guiding the distal end of the guiding and steering system to the cardiac tissue;
piercing the cardiac tissue using the piercing means, and delivering radio-frequency energy to the piercing means to form the transmural channel.
